(19) United States
(12) Patent Application Publication
(10) Pub. No.: US 2019/0323890 A1
(43) Pub. Date: Oct. 24, 2019

(54) IMAGING SENSOR WITH FILTER AND LENS ARRAY

(71) Applicant: KONINKLIJKE PHILIPS N.V., EINDHOVEN (NL)

(72) Inventor: SZILVESZTER CSEH JANDO, NAUGATUCK, CT (US)

(21) Appl. No.: 16/474,734

(22) PCT Filed: Dec. 28, 2017
(86) PCT No.: PCT/EP2017/084680

§ 371 Date: Jun. 28, 2019

Related U.S. Application Data
(60) Provisional application No. 62/442,588, filed on Jan. 5, 2017.

(51) Int. Cl. G01J 3/36; G01J 3/02; G01J 3/28; G01N 21/3504; A61M 16/10

(52) U.S. Cl. CPC G01J 3/36; G01J 3/0208; G01J 3/0229; G01J 3/0297; A61M 2205/3313; G01N 21/3504; A61M 16/1005; A61M 2016/103; A61M 2016/1035; G01J 3/2803

(57) ABSTRACT

An infrared imaging device including a filter array (110) comprising: (i) a plurality of spaced filter elements (116); (ii) a reflective coating (114), wherein the reflective coating is disposed on the filter array between each of the plurality of spaced filter elements; and (iii) a plurality of lenses (112), wherein each one of the plurality of lenses is aligned with a respective one of the plurality of spaced filter elements and is configured to focus electromagnetic radiation into a beam; and an imaging sensor array (120) spaced by a first distance from the filter array and comprising a plurality of pixel elements (122), wherein each of the plurality of pixel elements is aligned with a focused beam from a respective one of the plurality of spaced filter elements.

### FIELD OF THE INVENTION

The present disclosure is directed generally to methods and systems for real-time anesthetic and respiratory gas concentration monitoring using an imaging sensor array with an integrated lens array.

### BACKGROUND

Microbolometers are utilized in sensor arrays to measure the power of incident electromagnetic radiation via heating of a material having a temperature-dependent electrical resistance. The electromagnetic radiation hits and heats the material, changing its electrical resistance which can be detected and analyzed. Once exclusively used in military applications, microbolometers have now been established in the commercial arena and are thus experiencing a reduction in cost to manufacture and purchase. Current microbolometers, for example, typically have pixel counts starting at 80×60 (4800 pixels), with each pixel element comprising a temperature sensitive resistive material to create the microbolometer such as vanadium oxide or amorphous silicon. It is expected that pixel count and sensitivity will keep increasing as volume production increases with cost per unit decreasing. With greater pixel counts, more signal processing opportunities are possible.

Existing technology typically uses a spinning filter wheel with one thermal detector time-multiplexed, with the thermal detector seeing one specific band-pass filter element at a time as the wheel spins at some given speed. Measurements are serial and only one data channel provided. Other existing technologies use a multi-surfaced mirror to create several infrared beams traveling in different directions, each traveling to a band-pass filter with one thermal detector. Although several gases can be analyzed in parallel and in real-time, there is only one detector per data channel.

### SUMMARY OF THE INVENTION

Accordingly, there is a continued need in the art for an infrared imaging system, such as a microbolometer system, with greater sampling capacity and with an increased number of data channels in parallel, thereby providing oversampling of target gases and improved signal-to-noise performance.

The present disclosure is directed to inventive systems and methods for real-time respiratory gas concentration monitoring. Applied to an infrared imaging system, the inventive systems and methods comprise a mosaic filter/lens array mounted over a two-dimensional microbolometer sensor array to provide anesthetic and respiratory gas detection and concentration measurement functionality. The filter mosaic mounted above the microbolometer sensor array comprises infrared narrow band-pass filters, with each filter targeting the unique infrared absorption wavelength of a respiratory gas found under standard anesthetic procedures. The lens structure focuses the infrared energy under each band-pass infrared filter onto the pixel array of the microbolometer, thereby increasing signal, reducing optical or thermal cross-talk between adjacent band-pass filters, and improving the signal-to-noise ratio.

Generally, in one aspect, an infrared imaging device is provided. The device includes a filter array having: (i) a plurality of spaced filter elements; (ii) a reflective coating, where the reflective coating is disposed on the filter array between each of the plurality of spaced filter elements; and (iii) a plurality of lenses, where each one of the plurality of lenses is aligned with a respective one of the plurality of spaced filter elements and is configured to focus electromagnetic radiation into a beam; and an imaging sensor array spaced by a first distance from the filter array and comprising a plurality of pixel elements, wherein each of the plurality of pixel elements is aligned with a focused beam from a respective one of the plurality of spaced filter elements.

According to an embodiment, the imaging array comprises a microbolometer.

According to an embodiment, each of the pixel elements comprises a plurality of pixels. According to an embodiment, at least one of the plurality of pixel elements comprises at least a first plurality of pixels comprising a first material, and further comprises a second plurality of pixels comprising a second material.

According to an embodiment, each of the plurality of spaced filter elements comprises a narrow band-pass filter having a center wavelength corresponding to a target anesthetic or gas.

According to an embodiment, the center wavelength of each of the plurality of spaced filter elements does not overlap with the center wavelength of any other filter element.

According to an embodiment, the reflective coating is configured to minimize signal cross-talk between neighboring pixel elements.

According to an embodiment, the plurality of lenses is positioned on a second side of the filter array, the second side of the filter array facing the imaging sensor array. According to an embodiment, the plurality of lenses is positioned on a first side of the filter array, the first side of the filter array facing away from the imaging sensor array.

According to an embodiment, the plurality of pixel elements comprise at least a first pixel element comprising a first material, and a second pixel element comprising a second material.

According to an embodiment, at least one of the plurality of spaced filter elements is substantially opaque.

According to an embodiment, the center wavelength of a first one of the plurality of spaced filter elements is a first peak absorption wavelength of a target anesthetic or gas, and wherein the center wavelength of a second one of the plurality of spaced filter elements is a second peak absorption wavelength of the target anesthetic or gas.

According to an embodiment, the device is configured to detect a concentration of a target anesthetic and a concentration of a target respiratory gas.

According to an embodiment, the reflective coating comprises gold, platinum, titanium, palladium, nickel, copper, aluminum, and/or combinations thereof.

According to an aspect is an infrared imaging system. The infrared imaging system includes a filter array comprising: (i) a plurality of spaced filter elements; (ii) a reflective coating, wherein the reflective coating is disposed on the filter array between each of the plurality of spaced filter elements; and (iii) a plurality of lenses, wherein each of the plurality of spaced filter elements comprises a narrow band-pass filter having a center wavelength corresponding to a target anesthetic or gas, and further wherein each one of the plurality of lenses is aligned with a respective one of the plurality of spaced filter elements and is configured to focus electromagnetic radiation into a beam; and a microbolometer spaced by a first distance from the filter array and comprising a plurality of pixel elements, wherein each of the plurality of pixel elements is aligned with a focused beam from a respective one of the plurality of spaced filter elements.

According to an aspect is an infrared imaging device. The infrared imaging device includes a filter array comprising: (i) a plurality of spaced filter elements, wherein each of the plurality of spaced filter elements comprises a narrow band-pass filter having a center wavelength corresponding to a target, and further wherein at least one of the plurality of spaced filter elements is substantially opaque; (ii) a reflective coating, wherein the reflective coating is disposed on the filter array between each of the plurality of spaced filter elements; and (iii) a plurality of lenses, wherein each one of the plurality of lenses is aligned with a respective one of the plurality of spaced filter elements and is configured to focus electromagnetic radiation into a beam; and a microbolometer spaced by a first distance from the filter array and comprising a plurality of pixel elements, wherein each of the plurality of pixel elements is aligned with a focused beam from a respective one of the plurality of spaced filter elements; wherein the reflective coating is configured to minimize signal cross-talk between neighboring pixel elements.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a schematic representation of a microbolometer system, in accordance with an embodiment.

FIG. 2 is a schematic representation of a top view of a mosaic filter/focusing lens array, in accordance with an embodiment.

FIG. 3 is a schematic representation of a side view of a mosaic filter/focusing lens array, in accordance with an embodiment.

FIG. 4 is a schematic representation of a perspective side view of a mosaic filter/focusing lens array, in accordance with an embodiment.

FIG. 5 is a schematic representation of a perspective side view of a mosaic filter/focusing lens array, in accordance with an embodiment.

FIG. 6 is a schematic representation of a microbolometer system, in accordance with an embodiment.

FIG. 7 is a graph of pixel map of data generated by a microbolometer system, in accordance with an embodiment.

### DETAILED DESCRIPTION OF EMBODIMENTS

The present disclosure describes various embodiments of a system and method for monitoring gas concentration. More generally, Applicant has recognized and appreciated that it would be beneficial to provide an infrared imaging system configured to simultaneously measure the concentration of multiple anesthetic and respiratory gases using a plurality of data channels in parallel. Accordingly, the methods described or otherwise envisioned herein provide a microbolometer system comprising a mosaic filter/lens array mounted over a two-dimensional microbolometer sensor array to provide anesthetic and respiratory gas detection and concentration measurement functionality. The filter mosaic mounted above the microbolometer sensor array comprises infrared narrow band-pass filters, with each filter targeting the unique infrared absorption wavelength of respiratory gases found under standard anesthetic procedures. The lens structure focuses the infrared energy under each band-pass infrared filter onto the pixel array of the microbolometer, thereby increasing signal, reducing optical or thermal cross-talk between adjacent band-pass filters, and improving the signal-to-noise ratio. According to an embodiment, there is no need to cool the microbolometer, and it can function above ambient temperature in order to reject ambient temperature fluctuations. The microbolometer design can use a calibrated black body source to measure against, operating at much higher temperatures than found in an ambient environment, therefore providing more radiant power and thus a greater signal for a given gas species absorption coefficient.

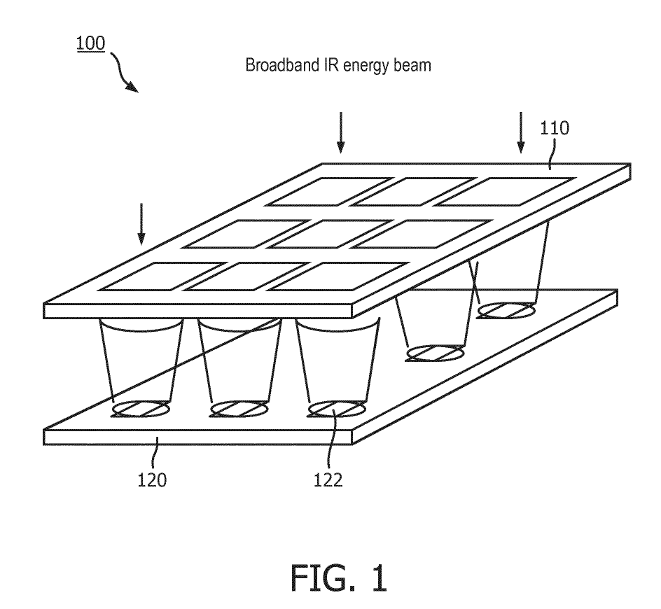

Referring to FIG. 1, in one embodiment, is an imaging sensor system 100 comprising a mosaic filter/focusing lens array 110 mounted above an imaging sensor array 120. The system is configured such that each lens in the mosaic filter/focusing lens array 110 focuses infrared energy onto a pixel or sub-pixel array 122 of the imaging sensor array. According to an embodiment, mosaic filter/focusing lens array 110 is mounted above imaging sensor array 120 during wafer level chip packaging assembly at an integrated circuit chip packaging foundry, although the system may be manufactured or assembled at any of a wide variety of other locations or stages in production, including but not limited to by a consumer purchasing individual components. The imaging sensor system 100 in the figure comprises a 3×3 array, although many other combinations are possible. According to an embodiment, imaging sensor array 100 is a microbolometer system, although the imaging sensor may be any other imaging sensor as described or otherwise envisioned herein.

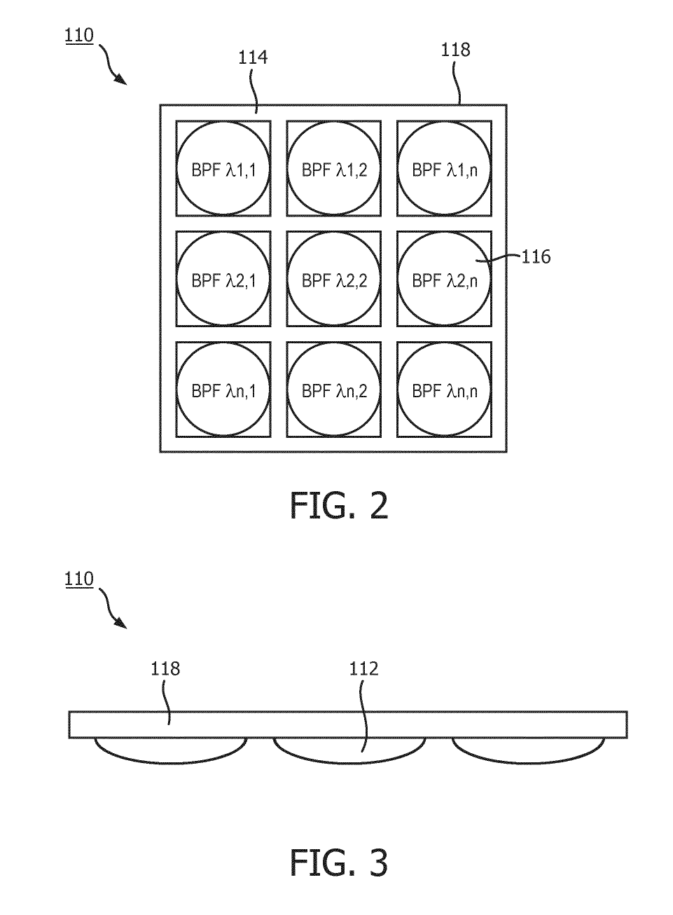

Referring to FIG. 2, in one embodiment, is a top view of a 3×3 mosaic filter/focusing lens array 110. According to an embodiment, mosaic filter/focusing lens array 110 comprises a mosaic filter array deposited on one surface side of a substrate material 118 suitable for transmission of long-wave infrared energy extending into the mid-wave infrared wavelengths from, for example, 2 μm to 15 μm. According to an embodiment, each infrared filter of each mosaic unit is a narrow band-pass filter (BPF) 116 with a center wavelength (λ_{c}) unique to each target anesthetic or respiratory gas species.

According to an embodiment, an infrared reflective coating grid 114 is deposited on the surface in order to mask off the filter mosaics to form a frame or grid about each band-pass filter mosaic to block passage of infrared energy in between adjacent mosaic elements. The reflective metal deposition creates a window frame structure to act as an aperture to minimize cross-talk between adjacent channels, and creates a dark zone between adjacent channels to form “dark pixels” that can be used for baseline subtraction of dark signal from the light signal for better signal to noise performance. For example, the reflective coating grid creates a shadow on the microbolometer pixel array between adjacent filter elements, thereby allowing for separation of the filter elements specifically associated with each sub-pixel group. This enables a determination and identification of the boundaries of each pixel sub-array.

According to an embodiment, infrared reflective coating 114 may comprise, for example, one or more materials, including but not limited to gold, platinum, titanium, palladium, nickel, copper, aluminum, and/or combinations thereof, among many other types of materials suitable for reflecting infrared energy. The reflective coating may be a layer of approximately 0.25 to 5 μm, although a layer thinner than 0.25 μm or thicker 5 μm is possible.

According to an embodiment, infrared reflective coating 114 can be deposited using physical vapor deposition or another thick layer deposition or electrochemical deposition process. According to an embodiment, the deposition can be performed by or in combination with a lithographing masking process.

According to an embodiment, each filter of each mosaic unit is a narrow band-pass filter (BPF) 116 with a center wavelength (λ_{c}) unique to a plurality of target anesthetics and/or respiratory gas species, including but not limited to nitrous oxide, desflurane, isoflurane, sevoflurane, halothane, enflutane, carbon dioxide, and/or one or more other gasses or anesthesia, including inhalational anesthetics. These gases typically have spectroscopic absorption bands in the mid-wave to long-wave infrared wavelengths from 2 to 15 μm, although other wavelengths are possible. The BPF's center wavelength (λ_{C}) and bandwidth can be selected as to not overlap in the wavelength of any of the other target gases.

As an example, a 3×3 filter mosaic array 110 would have a total of nine filter elements 116, such as in FIG. 1. According to this example, seven of the filter elements 116 can be gas-specific band-pass filter elements (such as for nitrous oxide, desflurane, isoflurane, sevoflurane, halothane, enflutane, and carbon dioxide), one of the filter elements 116 can be for background subtraction, and one of the filter elements 116 can be utilized to detect water vapor. The filter element 116 for background subtraction could comprise a reflective coating deposited on the element so that element is blocked, for example. This configuration might eliminate a need for a mechanical shutter or a continuously modulated infrared source.

According to an embodiment, the filter mosaic is constructed with a suitable substrate material as to minimize infrared energy transfer losses to the microbolometer pixel array. Substrate material examples could be silicon, germanium, zinc selenide, and/or GASIR, among many others. A preferred substrate material comprises wide infrared wavelength bandwidths with high transmittance for the target gas absorption wavelengths.

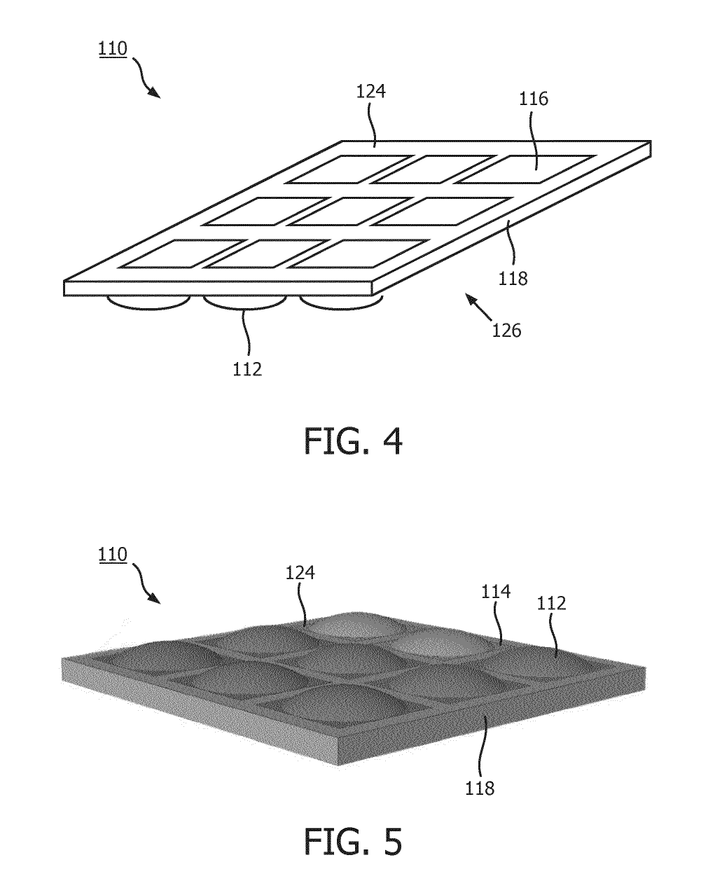

Referring to FIG. 3, in one embodiment, is a side view of a mosaic filter/focusing lens array 110. According to an embodiment, mosaic filter/focusing lens array 110 comprises a substrate material 118 suitable for transmission of infrared energy. Underneath each filter mosaic element 116 (not shown) found on the top side of the substrate material is a focusing lens 112. Accordingly, the filter array 110 comprises a substrate material 118 with a first surface comprising a plurality of spaced filter mosaic elements 116 and a second surface comprising a plurality of focusing lenses 112, where each filter mosaic element 116 is aligned with a respective one of the focusing lenses 112. Similarly, referring to FIG. 4 in one embodiment is a perspective side view of a filter array 110 comprising substrate material 118 with a first surface 124 comprising a plurality of spaced filter mosaic elements 116 and a second surface 126 comprising a plurality of focusing lenses 112, where each filter mosaic element 116 is aligned with a respective one of the focusing lenses 112.

Referring to FIG. 5 in one embodiment is a perspective side view of a filter array 110 comprising substrate material 118 with a first surface 124 comprising a plurality of spaced filter mosaic elements each with a focusing lens 112. In this embodiment, the focusing lens 112 is convex and is placed on the first surface 124 over the filter mosaic element. A reflective coating grid 114 is deposited on the surface in order to mask off the filter mosaics to form a frame or grid about each band-pass filter mosaic.

According to an embodiment, the band-pass filter material 116 is deposited on the filter mosaic substrate material 118 utilizing a micro-lithographic process, in which sets of masks along with chemical deposition processes are used to create a mosaic consisting of a plurality of square or rectangular areas, each with a specific infrared band-pass filter. According to an embodiment, the process of creating filter arrays is performed on a semiconductor wafer level scale, with the wafer size dependent upon the substrate material. Depending on the size of the wafer, for example, N number of filter arrays can be created per wafer to maximize the number of filter array units that can be created and thus reduce cost per unit. Although the spaced filter mosaic elements 116 are shown as being square or rectangular areas, they may be any other shape or size, including round, triangular, or any other shape or size.

According to another embodiment, the focusing lens 112 may be deposited on the filter mosaic substrate material 118 utilizing a micro-lithographic process or molding process, among other methods. According to another embodiment, the focusing lens 112 may be positioned on the second surface 126 of the filter array 110, on the first surface 124 of the filter array 110, or on both the first and second surfaces of the filter array. The lens structure 112 may be convex or plan-convex in order to produce a cone of thermal energy concentrated and/or focused on the microbolometer sensor array surface, as shown in FIG. 1. This increases signal, reduces optical or thermal cross-talk between adjacent band-pass filters, and improves the signal-to-noise ratio.

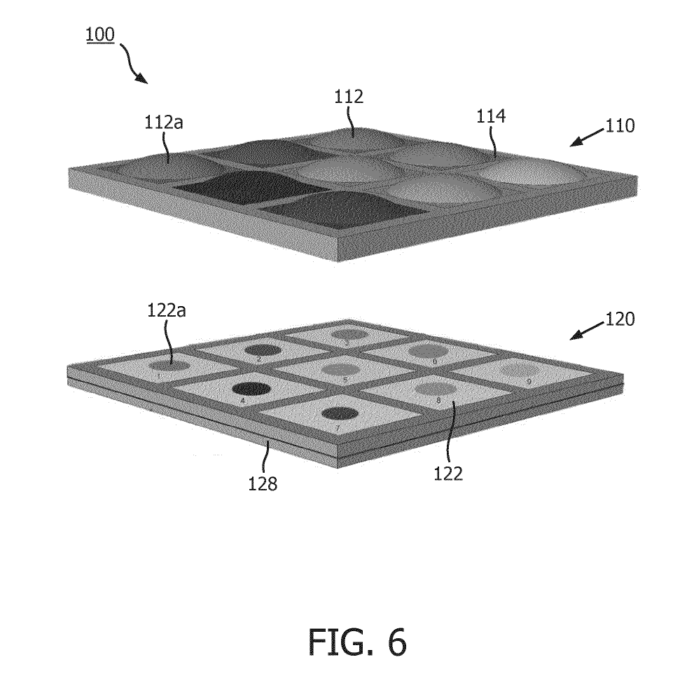

Referring to FIG. 6, in one embodiment, is a microbolometer system 100 comprising a mosaic filter/focusing lens array 110 mounted above a microbolometer sensor array 120. The system is configured such that each lens in the mosaic filter/focusing lens array 110 focuses infrared energy onto the microbolometer sensor array 120, comprising a plurality of pixel or sub-pixel arrays 122. According to an embodiment, each element 112 and/or 116 in the mosaic filter/focusing lens array 110 is mounted above a respective pixel or sub-pixel array 122 of the microbolometer sensor array 120. The filter/focusing lens array 110 of microbolometer system 100 also comprises a reflective coating grid 114 to mask off the filter mosaics to form a frame or grid to block passage of infrared energy in between adjacent array elements.

As shown in FIG. 6, the microbolometer system 100 comprises a 3×3 array, although many other combinations are possible. Each lens 112 in the filter array 110 passes a narrow bandwidth of LWIR energy to the corresponding individual microbolometer focal plane sensor element 122 directly beneath it, creating a circular spot of focused LWIR energy. The size of the spot can be large enough to utilize as much of the surface area of element 122, but preferably is not so great that it encompasses the entire area, leaving an area outside the focused spot with pixels in the dark which can be used for baseline subtraction. According to an embodiment, each microbolometer array sensor element can be of any n×n size, for instance a 30×30 pixel sensor with 900 sensor elements, or even a great order such as 80×80 with 6400 pixel sensors. In FIG. 6, for example, the filter array element 112a focuses the energy on the spot on the surface of the corresponding microbolometer sensor element 122a. The spot size is less than the total surface area of sensor array 122a, with only perhaps 50% exposed to the infrared energy, although many other percentages are possible, including much larger and much smaller, although less than the whole. Indeed, a remaining unexposed area of the sensor is in the dark due to the reflective metal grid 114 on the filter array 110. The dark pixels can be used for signal processing such as baseline subtraction.

According to an embodiment, each microbolometer array sensor element 122 can be fabricated on the same silicon die or can be individual silicon dies. A CMOS process to create the circuitry for each microbolometer array element can be used with MEMS fabrication to create the pixel arrays 122. The read out circuitry for the system 100 can be part of each microbolometer array element, which can be on the same silicon die or individual dies. According to an embodiment, in order to breakout electrical signals from each array 122, a second silicon wafer 128 can be bonded to the wafer containing the microbolometer array elements. This second wafer could serve as an interposer to allow signals to be reached at microbolometer arrays elements that are surrounded by microbolometer sensor array elements that are on the perimeter. For example, referring to FIG. 6, the central pixel arrays 122 might not be able to connect to external electronics without the use of an interposer layer beneath it.

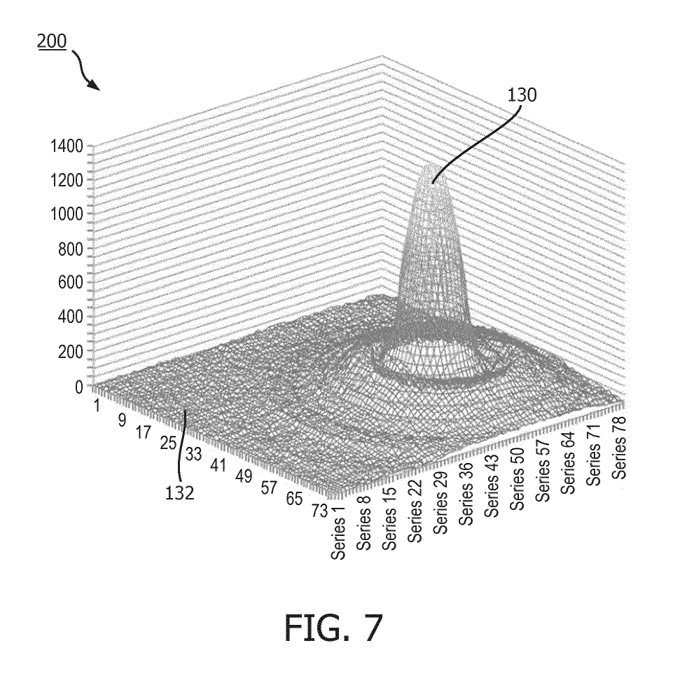

Referring to FIG. 7, in one embodiment, is a pixel map 200 of data generated by a microbolometer system 100. A convex lens structure 112 results in a cone of thermal energy concentrated and/or focused on the microbolometer sensor array surface, as shown in FIG. 1. This focused cone of thermal energy resulting from the convex or substantially convex lens structure produces a peak 130 of energy amid a background 132 of energy.

According to another embodiment, the sensor array 120 may be any other sensor configured to detect or measure the power of incident electromagnetic radiation. For example, the sensor array 120 may be a two-dimensional array comprising a thermopile, pyroelectric, thermistor, biomaterial microcantilever thermal sensor, and thermal diode. According to one embodiment, the sensor array comprises sensitivity from the mid-wave to long-wave infrared band, including from 2 μm to 15 μm. The ambient temperature operating microbolometer sensitivities can be specified as noise equivalent temperature difference (NETD) in the 20 mK to 100 mK range, with some technologies are pushing NETD's to about 3 mK, including bimaterial microcantilever thermal sensor arrays.

According to an embodiment, a custom microbolometer array 120 can be designed such that one or more of the pixel array 122 associated with the filter elements 116 can be divided into pixel subarrays 122. By breaking up a pixel array into a smaller sub-array, the pixel voltage gain, bias voltage, and offset voltages can be adjusted independently of one another. For example, some target gases may have high absorption peaks requiring less gain in the amplifier versus gases that have low absorption peaks requiring higher amplifier gains. However, most microbolometers array sensors typically have one global gain, bias, and offset adjustment available, which may sacrifice dynamic range and signal-to-noise ratio for some of the gases. By breaking the array into subarrays each with their own amplifier circuitry, more flexibility can be provided. For example, nitrous oxide have strong absorption at 16.949 um and would require a low gain to measure the absorption signal, while other anesthetic gases such as halothane will require a high gain setting. Amplifier gain in a microbolometer is set with an integrator circuit that collects charge, with a longer the integration time meaning higher gain. Longer integration times may reduce the frame rate of the sensor as opposed to low gain settings where higher frame rates can be achieved. For example, referring to FIG. 6, there can be nine different gain values, nine different bias voltages, and nine different frames rates to collect the data coming from the nine individual microbolometer sensor array elements 122.

Indeed, another advantage of sub-pixel arrays is that each can have their own read-out channel thus increasing the data read-out speed as opposed to one read-out channel for the entire pixel array. Faster read-out also can increase the frame rate of the sensor, thus sampling the gas samples faster improving the real-time measurements. Indeed, microbolometer pixel arrays can be read-out in sub-array areas, increasing the speed of data readout and reducing the amount of memory required for data storage and processing. For example, if only one or some of the anesthetic gases will be used at the same time, then only the section of the pixel array under the band-pass filter for that one or more gases need be read-out. The remainder of the array is either not read-out, or the non-relevant data is discarded.

Another advantage of individual microbolometer array sensor elements 122 rather than a single element is the ability to make a microbolometer with a plurality of different microbolometer sensor materials. Each different material used for a sensor will have different responsivities to different wavelengths of thermal energy when exposed to a black body thermal source. With individual microbolometer array sensors elements 122, it is possible to have a different microbolometer sensing material used for each of the specific elements of the microbolometer array. Referring to a 3×3 array with nine individual microbolometer sensor array elements 122, one of the nine elements could be a vanadium oxide-based element while another could be an amorphous silicon-based element to provide the best responsivity to thermal energy at the targeted bandwidth of infrared energy that those individual sensor elements would require to detect an anesthetic gas.

According to an embodiment, having a plurality of filters of different wavelengths provides numerous benefits, including the ability to use more than one wavelength for each target gas. For example, if using two or more wavelengths an additive measurement can be made to improve the signal-to-noise ratio of the measurement. For example, isoflurane has its strongest absorption peaks at 1167.5 cm⁻¹ and 1212 cm⁻¹. By providing two having filters centered about these two peaks, both can be used to calculate a total absorption value, thereby improving the signal-to-noise ratio.

According to an embodiment, with a microbolometer system 100 in which anesthetic gas concentration is measured simultaneous at a given frame rate and as sensitivity and the thermal time constant of microbolometer sensors continues to improve, it is possible to measure not only the gas concentration being supplied to the patient but the residual concentration of gas expired by the patient on a breath by breath basis, such as CO₂ monitoring in capnography. This allows an anesthesiologist to understand what's going in and what's coming out to determine how much was absorbed by the patient, for example.

According to an embodiment, the microbolometer system 100 can be calibrated at the factory and/or before, during or after a use of the system. For example, the system can be kept at a constant temperature, above ambient temperature, to prevent thermal drift of the sensor array. A sub-pixel array located underneath a reflective filter mosaic element, which blocks the energy, can be used for dark background subtraction with and without the infrared source turned on. A dead or bad pixel map of the array can then be created, and dead or bad pixels can be eliminated from the dataset calculations. According to an embodiment, the source energy can be adjusted to get an optimized balance of dynamic range and gain sensitivity for the active areas of the pixel array and cover the desired resolution for each gas species.

According to an embodiment, the microbolometer system 100 can be utilized as part of a larger system, such as an airway measurement system. Depending on the absorption coefficients for the target gases, the optical path length may be long, including 1 meter or more, so in order to shorten this path length there can be one or more optical bounces over a shorter path length within the system, thus creating an equivalent optical path length over a shorter physical distance.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. An infrared imaging device, comprising:
a filter array comprising: (i) a plurality of spaced filter elements; (ii) a reflective coating, wherein the reflective coating is disposed on the filter array between each of the plurality of spaced filter elements; and (iii) a plurality of lenses, wherein each one of the plurality of lenses is aligned with a respective one of the plurality of spaced filter elements and is configured to focus electromagnetic radiation into a beam; and
an imaging sensor array spaced by a first distance from the filter array and comprising a plurality of pixel elements, wherein each of the plurality of pixel elements is aligned with a focused beam from a respective one of the plurality of spaced filter elements.

2. The infrared imaging device of claim 1, wherein the imaging array comprises a microbolometer.

3. The infrared imaging device of claim 1, wherein each of the pixel elements comprises a plurality of pixels.

4. The infrared imaging device of claim 3, wherein at least one of the plurality of pixel elements comprises at least a first plurality of pixels comprising a first material, and further comprises a second plurality of pixels comprising a second material.

5. The infrared imaging device of claim 1, wherein each of the plurality of spaced filter elements comprises a narrow band-pass filter having a center wavelength corresponding to a target anesthetic or gas.

6. The infrared imaging device of claim 5, wherein the center wavelength of each of the plurality of spaced filter elements does not overlap with the center wavelength of any other filter element.

7. The infrared imaging device of claim 1, wherein the reflective coating is configured to minimize signal cross-talk between neighboring pixel elements.

8. The infrared imaging device of claim 1, wherein the plurality of lenses are positioned on a second side of the filter array, the second side of the filter array facing the imaging sensor array.

9. The infrared imaging device of claim 1, wherein the plurality of lenses are positioned on a first side of the filter array, the first side of the filter array facing away from the imaging sensor array.

10. The infrared imaging device of claim 1, wherein the plurality of pixel elements comprise at least a first pixel element comprising a first material, and a second pixel element comprising a second material.

11. The infrared imaging device of claim 1, wherein at least one of the plurality of spaced filter elements is substantially opaque.

12. The infrared imaging device of claim 1, wherein the center wavelength of a first one of the plurality of spaced filter elements is a first peak absorption wavelength of a target anesthetic or gas, and wherein the center wavelength of a second one of the plurality of spaced filter elements is a second peak absorption wavelength of the target anesthetic or gas.

13. The infrared imaging device of claim 1, wherein the device is configured to detect a concentration of a target anesthetic and a concentration of a target respiratory gas.

14. The infrared imaging device of claim 1, wherein the reflective coating comprises gold, platinum, titanium, palladium, nickel, copper, aluminum, and/or combinations thereof.

15. An infrared imaging system, the system comprising:
a filter array comprising: (i) a plurality of spaced filter elements; (ii) a reflective coating, wherein the reflective coating is disposed on the filter array between each of the plurality of spaced filter elements; and (iii) a plurality of lenses, wherein each of the plurality of spaced filter elements comprises a narrow band-pass filter having a center wavelength corresponding to a target anesthetic or gas, and further wherein each one of the plurality of lenses is aligned with a respective one of the plurality of spaced filter elements and is configured to focus electromagnetic radiation into a beam; and
a microbolometer spaced by a first distance from the filter array and comprising a plurality of pixel elements, wherein each of the plurality of pixel elements is aligned with a focused beam from a respective one of the plurality of spaced filter elements.

16. The infrared imaging system of claim 15, wherein each of the pixel elements comprises a plurality of pixels.

17. The infrared imaging system of claim 15, wherein the plurality of lenses are positioned on a second side of the filter array, the second side of the filter array facing the imaging sensor array.

18. The infrared imaging system of claim 15, wherein the plurality of lenses are positioned on a first side of the filter array, the first side of the filter array facing away from the imaging sensor array.

19. The infrared imaging system of claim 15, wherein the center wavelength of a first one of the plurality of spaced filter elements is a first peak absorption wavelength of a target anesthetic or gas, and wherein the center wavelength of a second one of the plurality of spaced filter elements is a second peak absorption wavelength of the target anesthetic or gas.

20. An infrared imaging device, the system comprising:
a filter array comprising: (i) a plurality of spaced filter elements, wherein each of the plurality of spaced filter elements comprises a narrow band-pass filter having a center wavelength corresponding to a target, and further wherein at least one of the plurality of spaced filter elements is substantially opaque; (ii) a reflective coating, wherein the reflective coating is disposed on the filter array between each of the plurality of spaced filter elements; and (iii) a plurality of lenses, wherein each one of the plurality of lenses is aligned with a respective one of the plurality of spaced filter elements and is configured to focus electromagnetic radiation into a beam; and
a microbolometer spaced by a first distance from the filter array and comprising a plurality of pixel elements, wherein each of the plurality of pixel elements is aligned with a focused beam from a respective one of the plurality of spaced filter elements;
wherein the reflective coating is configured to minimize signal cross-talk between neighboring pixel elements.

21. The infrared imaging device of claim 20, wherein each of the pixel elements comprises a plurality of pixels.
